(19) United States
(12) Patent Application Publication
(10) Pub. No.: US 2013/0108570 A1
(43) Pub. Date: May 2, 2013

(54) Antiperspirant Soft Solid Compositions Comprising A Wax Blend

(71) Applicant: The Procter & Gamble Company, Cincinnati, OH (US)

(72) Inventors: David Arthur Sturgis, Cincinnati, OH (US); Phi Van Chu, Cincinnati, OH (US); Steven Michael Wujek, Loveland, OH (US)

(73) Assignee: THE PROCTER & GAMBLE COMPANY, Cincinnati, OH (US)

(21) Appl. No.: 13/661,060

(22) Filed: Oct. 26, 2012

Related U.S. Application Data
(60) Provisional application No. 61/552,058, filed on Oct. 27, 2011.

(51) Int. Cl. A61K 9/42; A61Q 15/00

(52) U.S. Cl. 424/65

(57) ABSTRACT

An antiperspirant soft solid composition includes a wax blend with glyceryl tribehenate, a first wax additive, and a second wax additive.

### TECHNICAL FIELD

The present disclosure generally relates to antiperspirant soft solid compositions and methods relating thereto.

### BACKGROUND

Antiperspirant soft solids, which can be applied by conventional means or packaged in topical dispensers, have become increasingly more popular as an effective alternative to antiperspirant sprays and solid sticks. While these soft solids can provide perspiration and odor control, such soft solids can have issues with stability and sometimes have issues with syneresis. As such, improvements are needed with respect to soft solid compositions and methods relating thereto.

### SUMMARY

An antiperspirant soft solid composition comprising a wax blend, the wax blend comprising: a) from about 0.1% to about 6.0% of glyceryl tribehenate, by weight of the antiperspirant soft solid composition; b) from about 0.1% to about 5.0% of a first wax additive, by weight of the antiperspirant soft solid composition, the first wax additive comprises one of a monoglyceride, a polymeric thickener, or an inorganic thickener; and c) from about 0.1% to about 5.0% of a second wax additive, by weight of the antiperspirant soft solid composition, wherein the second wax additive comprises one of the remaining monoglyceride, polymeric thickener, or inorganic thickener, such that the first wax additive and the second wax additive are different; wherein the antiperspirant soft solid composition is substantially free of a C₁₈-C₃₆ triglyceride and exhibits a static yield stress value from about 100 Pa to about 3,000 Pa.

An antiperspirant soft solid composition, comprising: 1) from about 3% to about 8%, by weight of the composition, of a wax blend comprising a) from about 1% to about 6%, by weight of the composition, of glyceryl tribehenate; b) from about 0.5% to about 2.5%, by weight of the composition, of ozokerite; c) from about 0.3% to about 3.5%, by weight of the composition, of polymethylene; and 2) from about 55% to about 75%, by weight of the composition, of a liquid carrier; wherein the composition is substantially free of a C₁₈-C₃₆ triglyceride and microcrystalline wax.

An anhydrous soft solid antiperspirant composition, comprising: 1) from about 3% to about 6.5%, by weight of the composition, of a wax blend consisting essentially of: a) from about 1.5% to about 2.5%, by weight of the composition, of glyceryl tribehenate; b) from about 1.5% to about 2.5%, by weight of the composition, of ozokerite; c) from about 0.75% to about 1.25%, by weight of the composition, of polymethylene with a melting point of about 65° C. to about 70° C.; and 2) from about 55% to about 75%, by weight of the composition, of a liquid carrier.

### DETAILED DESCRIPTION

### I. Definitions

As used herein, the following terms shall have the meaning specified thereafter:

“Ambient” refers to surrounding conditions at about one atmosphere of pressure, 50% relative humidity and about 25° C.

“Anhydrous” refers to compositions and/or components which are substantially free of added or free water.

“Antiperspirant composition” refers to antiperspirant compositions, deodorant compositions, and the like. For example, antiperspirant creams, gels, and soft solid sticks.

“Soft solid” refers to a composition with a static yield stress of about 200 Pa to about 1,300 Pa.

“Substantially free of” refers to about 2% or less, about 1% or less, or about 0.1% or less of a stated ingredient. “Free of” refers to no detectable amount of the stated ingredient or thing.

### II. Antiperspirant Soft Solid Compositions

Some formulations of antiperspirant soft solids have been unsuccessful and others, while successful, are not ideal due to a lack of physical stability in the cream. This instability can result in solvent syneresis (weeping of solvent from the cream matrix), which can occur during packaging, storage, shipping, or even application of the cream. Product instability can be minimized or eliminated in these soft solids by simply formulating the product into a harder, more conventional antiperspirant solid stick, but many consumers prefer the lower residue associated with the soft solid form. Soft solids are often applied with an applicator device having a perforated cap through which the soft solid composition can be extruded and applied to the skin. Antiperspirant solid sticks are too hard to be extruded through most perforated caps, and typically result in higher visible residue on the skin than soft solids.

Currently, there are antiperspirant soft solids that can deliver improved cosmetics, product stability, and reduced solvent syneresis. Known methods for creating these improved soft solids have relied upon the addition of C₁₈-C₃₆ triglycerides, such as SYNCROWAX® HGL-C, as a vital component. But currently, the long-term availability of SYNCROWAX® HGL-C and related waxes is in doubt, and a replacement wax or blend of waxes is desired such that consumers can be provided with the benefits of soft solids having improved cosmetics, product stability, and reduced solvent syneresis even if SYNCROWAX® HGL-C is no longer available.

Initially, single wax drop in replacements were tried as a substitute for SYNCROWAX® HGL-C, but these surprisingly did not provide the properties, like stability and/or rheology, that were desired for a consumer acceptable product. Even some dual wax samples were unsuccessful. See, for example, Comparative Formulas 1 and 2 from Table 5 below which are both dual wax systems. Comparative Formulas 1 and 2 had Weeping Scores of 1.09 and 0.87, respectively, which are well above the commercially available product, Commercial Example 1 with HGL-C which had a Weeping score of 0.46, and thus unacceptable as an alternate formulation from a stability point of view.

Surprisingly, the present inventors have discovered that the replacement of C₁₈-C₃₆ triglycerides with an inventive wax blend results not only in a consumer acceptable product, but provides an improvement in syneresis versus a C₁₈-C₃₆ triglyceride composition. This is demonstrated in the data which shows that a currently marketed antiperspirant product with a C₁₈-C₃₆ triglyceride (Commercial Example 1) has a significantly higher Vacuum Weeping Score (0.2760 g) than Inventive Example 2 (0.0180 g), Inventive Example 4 (0.0803 g), and Inventive Example 6 (0.0085 g). Unlike the Weeping Scores above, Vacuum Weeping is done under pressure and is a more rigorous evaluation of stability. A higher weeping score in the Vacuum Weeping test means the composition showed more weeping under a low pressure. While the Vacuum Weeping Score was only measured for Inventive Formulas 2, 4, and 6, it is believed the scores for all Inventive Formulations in the examples would have a weeping score of 0.10 g or less. The methods for measuring the Weeping Score and Vacuum Weeping Score are set out below.

In addition, it is believed the wax blend can also alter the rheology of the inventive compositions and can also contribute to enhanced stability, especially during dispensing. This is due, at least in part, to an increase in high shear viscosity. See, for example, Table 6 below which shows Inventive Examples 1 and 2 have a higher high shear viscosity than Commercial Example 1. A higher high shear viscosity means the composition is more robust when under greater stress which can help reduce syneresis. This improvement helps provide a more positive experience for the consumer as it helps alleviate wet and/or greasy feelings during use because of syneresis.

TABLE 6

| | Commercial | Inventive | Inventive |
|---|---|---|---|
| | Example 1 | Example 1 | Example 2 |
| High Shear | 6,378 | 16,100 | 8,858 |
| Rheology (@ 10 | | | |
| inverse seconds) | | | |

The dramatic reduction in the Vacuum Weeping score, and thus greater stability of the composition, also allows for the reduction in the total amount of wax in the composition. Traditionally, wax is used as a structurant to stabilize soft solid compositions to help minimize weeping. Most soft solids use wax in the range of 5 to 8%, by weight of the soft solid composition. The improvement in stability of the inventive formulas allows them to have wax levels of about 3 to about 6%, by weight of the composition, while maintaining an acceptable stability. Since higher wax levels can have negative impacts on characteristics of the composition, like active release, there is an advantage to being able to use lower wax levels.

Thus, as can be seen above, there are a multitude of advantages with the inventive wax blends.

A. Wax Blends

An antiperspirant soft solid composition can include a wax blend. Wax blends can help control the rheology profile, provide a desired viscosity, texture and/or product hardness, or to otherwise suspend any dispersed solids or liquids within the antiperspirant soft solid composition.

Wax blends can be present in an amount of from about 0.1%, from about 1%, or from about 3%; to about 12%, to about 8.5%, to about 8%, to about 7.5%, to about 6.5%, to about 6.0%, to about 5.5%, to about 5%, to about 4.5%, to about 4.0%, or any combination thereof, by weight of an antiperspirant soft solid composition. Concentrations and types of materials selected for use in the wax blend can vary depending on a desired product form.

Suitable materials for use in the wax blend can include waxes and wax additives. Suitable waxes can include triglycerides (e.g., glyceryl tribehenate), excluding C₁₈-C₃₆ triglycerides; hydrogenated castor oil (castor wax); bayberry; beeswax; candelilla; carnauba; ceresin; montan; microcrystalline waxes; other waxes or wax-like materials having a melting point of about 65° C. or greater, and combinations thereof. Other suitable high melting point waxes are described in U.S. Pat. No. 4,049,792, Elsnau, issued Sep. 20, 1977. The composition may be substantially free of or free of castor wax. The composition may also be substantially free of or free of microcrystalline wax.

Non-limiting examples of suitable tryiglycerides can include glyceryl tribehenate such as SYNCROWAX® HRC(SYNCROWAX® available from Croda, Inc.), tristearin, hydrogenated vegetable oil, trihydroxysterin (Thixcin® R, available from Rheox, Inc.), rape seed oil, castor wax, fish oils, and tripalmitin. A wax blend can include, for example, a glyceryl tribehenate, a first wax additive, and a second wax additive. Glyceryl tribehenate can be included, for example, at an amount of from about 0.1% to about 6.0%, from about 0.5% to about 5.5%; from about 1.0% to about 5%; from about 1.5% to about 4.5%; from about 1.5% to about 2.5%, from about 2.0% to about 4%, or any combination thereof, by weight of an antiperspirant soft solid composition.

An antiperspirant soft solid composition can include a wax blend substantially free of a C₁₈-C₃₆ triglyceride (e.g., SYNCROWAX® HGL-C). Further, a wax blend can be substantially free of a combination of glyceryl tribehenate and a C₁₈-C₃₆ triglyceride (e.g., SYNCROWAX® HGL-C).

A wax blend can include one or more wax additives. Each wax additive may be present in an amount from about 0.1% to about 5%; from about 0.3% to about 3.8%; from about 0.3 to about 3.5%, from about 0.5% to about 3.5%; from 0.5% to about 2.5%, from about 0.75% to about 1.25%, from about 0.8% to about 3%; from about 0.9% to about 2.5%; from about 1% to about 2.3%; from about 1.0% to about 1.5%, from about 1.2% to about 2%; from about 1.5% to about 1.7%; from about 1.5% to about 2.5%, or any combination thereof, by weight of an antiperspirant soft solid composition.

Wax additives can include, but are not limited to monoglycerides, such as sucrose polybehenate; polymeric thickeners such as polymethylene and polyethylene; inorganic thickeners such as ozokerite, and combinations thereof.

Suitable wax additives can include monoglycerides. Such monoglycerides may include, for example, sucrose polybehenate.

Suitable wax additives may also include polymeric thickeners. Such polymeric thickeners may include, for example, polymethylene, polyethylene, and combinations thereof. Some suitable polyethylenes may have a melting point from about 65° C. to about 75° C. Examples of suitable polyethylenes include those with a melting point from about 60° C. to about 95° C.

Additional suitable wax additives may also include inorganic thickeners. These inorganic thickeners may include, for example, clays, colloidal pyrogenic silica pigments, and combinations thereof. For example, colloidal pyrogenic silica pigments such as CAB-O-SIL®, a submicroscopic particulated pyrogenic silica, can be used. Suitable clay thickening agents can include montmorillonite clays such as bentonites, hectorites, and colloidal magnesium aluminum silicates. These and other suitable clays can be hydrophobically treated, and when so treated, can be used in combination with a clay activator. Non-limiting examples of suitable clay activators can include propylene carbonate, ethanol, and combinations thereof. Additionally, an inorganic thickening agent such as ozokerite can be used in a wax blend.

Suitable wax additives that can be included in an antiperspirant soft solid composition can include gelling materials. Such gelling materials can include, for example, fatty acid gellants, salts of fatty acids, hydroxyl acids, hydroxyl acid gellants, esters and amides of fatty acid or hydroxyl fatty acid gellants, cholesterolic materials, dibenzylidene alditols, lanolinolic materials, fatty alcohols, inorganic materials such as clays or silicas, other amide or polyamide gellants, and mixtures thereof. Additional suitable gelling materials can include fatty acid gellants such as fatty acid and hydroxyl or alpha hydroxyl fatty acids, having from about 10 to about 40 carbon atoms, and ester and amides of such gelling materials. Non-limiting examples of such gelling materials can include, but are not limited to 12-hydroxystearic acid, including esters and amides thereof, 12-hydroxylauric acid, 16-hydroxyhexadecanoic acid, behenic acid, eurcic acid, stearic acid, caprylic acid, lauric acid, isostearic acid, and combinations thereof.

Other suitable gelling materials can include amide gellants such as disubstituted or branched monoamide gellants, monosubstituted or branched diamide gellants, triamide gellants, and combinations thereof, including n-acyl amino acid derivatives such as n-acyl amino acid amides, n-acyl amino acid esters prepared from glutamic acid, lysine, glutamine, aspartic acid, and combinations thereof. Other suitable gelling materials (including amide gelling materials) are described in U.S. Pat. Nos. 5,429,816; 5,840,287; and 5,902,570.

Still other suitable gelling materials can include fatty alcohols having about 8 carbon atoms or more, about 12 carbon atoms or more, but no more than about 40 carbon atoms, no more than about 30 carbon atoms, or no more than about 18 carbon atoms. For example, fatty alcohols can include, but are not limited to cetyl alcohol, myristyl alcohol, stearyl alcohol and combinations thereof.

Some examples of suitable wax blends can include a wax blend comprising from about 4.5% to about 5.0% of glyceryl tribehenate, from about 1.0% to about 1.5% of a monoglyceride, and from about 0.5% to about 1.0% of a polymeric thickener, by weight of an antiperspirant soft solid composition; a wax blend can comprise from about 2.2% to about 2.5% of glyceryl tribehenate, from about 2.0% to about 2.5% of a polymeric thickener, and from about 0.9% to about 1.0% of an inorganic thickener, by weight of an antiperspirant soft solid composition; a wax blend can comprise from about 1.7% to about 4.5% of glyceryl tribehenate, from about 0.9% to about 2.9% of a polymeric thickener, and from about 0.3% to about 1.0% of an inorganic thickener, by weight of an antiperspirant soft solid composition; a wax blend can comprise from about 3.0% to about 3.5% of a polymeric thickener and from about 0.5% to about 1.0% of an inorganic thickener, by weight of an antiperspirant soft solid composition.

Additional examples of suitable wax blends include glyceryl tribehenate, ozokerite, and polyethylene; glyceryl tribehenate, ozokerite, and polymethylene; glyceryl tribehenate, sucrose polybehenate, and polyethylene; glyceryl tribehenate and sucrose polybehenate; glyceryl tribehenate and polyethylene; glyceryl tribehenate and polymethylene; glyceryl tribehante, polyethylene, polymethylene, cator wax, and ozokerite; and glyceryl tribehenate and polymethylene; glyceryl tribehante, polyethylene, polymethylene, and ozokerite. The wax blend may also consist essentially of any of these noted examples.

B. Liquid Carrier

An antiperspirant soft solid composition can include a liquid carrier. Suitable liquid carriers can include, but are not limited to, topically safe and effective organic, silicone-containing or fluorine-containing, volatile or non-volatile, polar or non-polar liquid carriers. In one embodiment, the liquid carrier can be liquid under ambient conditions, and can include one or more liquid carrier materials provided that any such combination of materials is in liquid form under ambient conditions. Depending on a type of product form desired, concentrations of the liquid carrier in antiperspirant soft solid compositions can typically range from about 10%, from about 30%, from about 55%, to about 75%, to about 90%, or any combination thereof, by weight of an antiperspirant soft solid composition.

Non-limiting examples of suitable liquid carriers can include C₁ to C₂₀ monohydric alcohols, i.e., C₂ to C₈ monohydric alcohols; C₂ to C₄₀ dihydric or polyhydric alcohols, i.e., C₂ to C₂₀ dihydric or polyhydric alcohols; alkyl ethers of all such alcohols, i.e. C₁-C₄ alkyl ethers; and polyalkoxylated glycols, i.e. propylene glycols and polyethylene glycols having from 2 to 30 repeating alkoxylate (e.g., ethoxylate or propoxylate) groups and polyglycerols having from 2 to 16 repeating glycerol moieties; and derivatives and mixtures thereof.

Specific examples of such alcohol liquid carriers can include propylene glycol, hexylene glycol, dipropylene glycol, tripropylene glycol, glycerin, propylene glycol methyl ether, dipropylene glycol methyl ether, ethanol, n-propanol, n-butanol, t-butanol, 2-methoxyethanol, 2-ethoxyethanol, ethylene glycol, isopropanol, isobutanol, 1,4-butylene glycol, 2,3-butylene glycol, trimethylene glycol, 1,3-butanediol, 1,4,-butanediol, propylene glycol monoisostearate, PPG-3 myristyl ether, PEG-4 (also known as PEG-200), PEG-8 (also known as PEG-400), 1,2, pentanediol, PPG-14 butylether, dimethyl isosorbide, and combinations thereof. Other similar but suitable solvents for use as liquid carriers can be described, for example, in U.S. Pat. No. 4,781,917, issued to Luebbe et al., Nov. 1, 1998, U.S. Pat. No. 5,643,558, issued to Provancal et al., Jul. 1, 1997, U.S. Pat. No. 4,816,261, issued to Luebbe et al., Mar. 28, 1989, and EP 404 533 A1, published Dec. 27, 1990 by Smith et al.

In one embodiment, an antiperspirant soft solid composition can include a silicone liquid carrier. A concentration of the silicone liquid carrier may range from about 10% or from about 15% of a silicone liquid carrier, by weight of the composition, to about 90% or to about 65% of a silicone liquid carrier, by weight of the composition. Suitable silicone liquid carriers can include volatile or non-volatile silicones.

Non-limiting examples of suitable silicone liquid carriers for use herein can include volatile silicones described in Todd et al., “Volatile Silicone Fluids for Cosmetics”, Cosmetics and Toiletries, 91:27-32 (1976). Suitable amongst these volatile silicones can include cyclic silicones having from about 3 or from about 4 to about 7 or to about 6, silicon atoms. Suitable silicon carriers can include those which can conform to a formula:

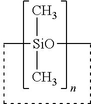

wherein n can be from about 3, from about 4 or from about 5 to about 7 or to about 6. Such volatile cyclic silicones can have a viscosity value of about 10 centistokes or less. Other suitable silicone liquid carriers for use herein can include volatile and nonvolatile linear silicones which conform to a formula:

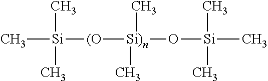

wherein n is greater than or equal to 0, Such volatile linear silicone materials can have viscosity values of about 5 centistokes or less at 25° C. Non-volatile linear silicone materials can have viscosity values of about 5 centistokes or greater at 25° C.

Suitable volatile silicones for use herein can include, but are not limited to, hexamethyldisiloxane; Silicone Fluids SF-1202 and SF-1173 (commercially available from G.E. Silicones); Dow Corning 244, Dow Corning 245, Dow Corning 246, Dow Corning 344, and Dow Corning 345, (commercially available from Dow Corning Corp.); Silicone Fluids SWS-03314, SWS-03400, F-222, F-223, F-250, and F-251 (commercially available from SWS Silicones Corp.); Volatile Silicones 7158, 7207, 7349 (available from Union Carbide); Masil SF-V™ (available from Mazer); and mixtures thereof.

Suitable non-volatile linear silicones for use herein can include, but are not limited to, Rhodorsil Oils 70047 available from Rhone-Poulenc; Masil SF Fluid available from Mazer; Dow Corning 200 and Dow Corning 225 (available from Dow Corning Corp.); Silicone Fluid SF-96 (available from G.E. Silicones); Velvasil™ and Viscasil™ (available from General Electric Co.); Silicone L-45, Silicone L-530, and Silicone L-531 (available from Union Carbide); and Siloxane F-221 and Silicone Fluid SWS-101 (available from SWS Silicones).

Other suitable non-volatile silicone liquid carriers for use in antiperspirant soft solid compositions can include, but are not limited to, non-volatile silicone emollients such as polyalkylarylsiloxanes, polyestersiloxanes, polyethersiloxane copolymers, polyfluorosiloxanes, polyaminosiloxanes, and combinations thereof. Such non-volatile silicone liquid carriers can have viscosity values of less than about 100,000 centistokes, less than about 500 centistokes, or from about 1 centistokes to about 200 centistokes or to about 50 centistokes, as measured under ambient conditions.

Other suitable liquid carriers for use in antiperspirant soft solid compositions can include, but are not limited to, organic liquid carriers such as mineral oil, petrolatum, isohexadecane, isododecane, various other hydrocarbon oils, and mixtures thereof. In one embodiment, mineral oil and branched chain hydrocarbons having from about 4 or from about 6 carbon atoms to about 30 or to about 20 carbon atoms can be suitable liquid carriers. Specific non-limiting examples of suitable branched chain hydrocarbon oils can include isoparaffins available from Exxon Chemical Company as Isopar C™ (C₇-C₈ Isoparaffin), Isopar E™ (C₈-C₉ Isoparaffin), Isopar G™ (C₁₀-C₁₁ Isoparaffin), Isopar H™ (C₁₁-C₁₂ Isoparaffin), Isopar L™ (C11-C13 Isoparaffin), Isopar M™ (C₁₃-C₁₄ Isoparaffin), and combinations thereof. Other non-limiting examples of suitable branched chain hydrocarbons can include Permethyl™ 99A (isododecane), Permethyl™ 102A (isoeicosane), Permethyl™ 101A (isohexadecane), and combinations thereof. The Permethyl™ series are available from Preperse, Inc., South Plainfield, N.J., U.S.A. Other non-limiting examples of suitable branched chain hydrocarbons can include petroleum distillates such as those available from Phillips Chemical as Soltrol™ 130, Soltrol™ 170, and those available from Shell as Shell Sol 70, −71, and −2033, and mixtures thereof.

Suitable organic liquid carriers can include a Norpar™ series of paraffins available from Exxon Chemical Company as Norpar™ 12, −13, and −15; octyldodecanol; butyl stearate; diisopropyl adipate; dodecane; octane; decane; C₁-C₁₅ alkanes/cycloalkanes available from Exxon as Exxsol™ D80; C₁₂-C₁₅ alkyl benzoates available as Finsolv-TN™ from Finetex; and mixtures thereof. Other suitable liquid carriers can include benzoate co-solvents, cinnamate esters, secondary alcohols, benzyl acetate, phenyl alkane, and combinations thereof.

Antiperspirant soft solid compositions can be formulated as aqueous or anhydrous compositions. Aqueous soft solid compositions can comprise from about 10% or from about 15% water, by weight of the composition, to about 75%, to about 60%, or to about 50% water, by weight of the composition. Anhydrous soft solid compositions may comprise about 2% or less, about 1% or less, about 0.5% or less, or zero percent water, by weight of the composition.

C. Antiperspirant Actives

An antiperspirant soft solid composition can also contain an antiperspirant active. Concentrations of an antiperspirant active can range from about 0.5% to about 60%, by weight of the composition; in certain embodiments an antiperspirant active can range from about 1% to about 35%, by weight of the composition; in certain embodiments an antiperspirant active can range from about 5% to about 25%, by weight of the composition. Such weight percentages can be calculated on an anhydrous metal salt basis exclusive of water and any complexing agents such as, for example, glycine and glycine salts. The antiperspirant active as formulated in the composition can be in a form of dispersed particulate solids having an average particle size or equivalent diameter of about 100 microns or less, about 20 microns or less, about 10 microns or less.

The antiperspirant actives can include any compound, composition, or other material having antiperspirant activity. The antiperspirant actives can include astringent metallic salts. In particular, the antiperspirant actives can include inorganic and organic salts of aluminum, zirconium and zinc, as well as mixtures thereof. Antiperspirant active examples can include, but are not limited to, aluminum-containing and/or zirconium-containing salts or materials, such as aluminum halides, aluminum chlorohydrate, aluminum hydroxyhalides, zirconyl oxyhalides, zirconyl hydroxyhalides, and mixtures thereof.

In one embodiment, aluminum salts can include those that conform to a formula:

Al₂(OH)ₐCl_{b}.×H₂O

wherein a is from about 0 to about 5; a sum of a and b is about 6; x is from about 1 to about 8; where a, b, and x can have non-integer values. For example, aluminum chlorohydroxides referred to as “¾ basic chlorohydroxide,” wherein a is about 4.5; “⅚ basic chlorohydroxide”, wherein a=5; and “⅔ basic chlorohydroxide”, wherein a=4 can be used. Processes for preparing aluminum salts are disclosed in U.S. Pat. No. 3,887,692, issued to Gilman on Jun. 3, 1975; U.S. Pat. No. 3,904,741, issued to Jones et al. on Sep. 9, 1975; and U.S. Pat. No. 4,359,456 issued to Gosling et al. on Nov. 16, 1982. A general description of such aluminum salts can also be found in Antiperspirants and Deodorants, Cosmetic Science and Technology Series Vol. 20, 2nd edition, edited by Karl Laden. Mixtures of aluminum salts are described in British Patent Specification 1,347,950, filed in the name of Shin et al. and published Feb. 24, 1974.

In one embodiment, zirconium salts can include those which conform to a formula:

ZrO(OH)₂₋ₐClₐ.×H₂O

wherein a is from about 0.5 to about 2; x is from about 1 to about 7; where a and x can both have non-integer values. Such zirconium salts are described in Belgian Patent 825,146, issued to Schmitz on Aug. 4, 1975. In one embodiment, antiperspirant soft solid compositions can include zirconium salt complexes that additionally contain aluminum and glycine, commonly known as “ZAG complexes.” Such complexes can contain aluminum chlorohydroxide and zirconyl hydroxy chloride conforming to formulas as set forth above. Such ZAG complexes are described in U.S. Pat. No. 4,331,609, issued to Orr on May 25, 1982 and U.S. Pat. No. 4,120,948, issued to Shelton on Oct. 17, 1978.

In one embodiment, an antiperspirant active can be employed at a concentration level of from about 5% to about 19%, by weight of the composition on an anhydrous basis. Other concentration levels can be contemplated for antiperspirant soft solid compositions, and accordingly, appended claims that do not recite a concentration level should not be interpreted to be limited to from about 5% to about 19%.

In one embodiment, a type and concentration of antiperspirant active can be chosen such that the antiperspirant soft solid composition can exhibit an Antiperspirant Efficacy Index of about 1.19 or less, about 1.0 or less, or about 0.9 or less. A methodology for measuring the Antiperspirant Efficacy Index is described in U.S. Pat. No. 6,352,688, issued to Scavone et al. on Mar. 5, 2002. The Antiperspirant Efficacy Index is calculated as a weight ratio of an amount (mg) of sweat collected from a control treatment side of a participant to an amount of sweat collected from a test product treatment side of that same participant. As used herein, and in accordance with the methodology, “Antiperspirant Efficacy Index” refers to a 3-day and/or a 10-day Antiperspirant Efficacy Index.

Antiperspirant soft solid compositions can alternatively or additionally include a deodorant active. Suitable deodorant actives can be selected from the group consisting of antimicrobial agents (e.g., bacteriocides, fungicides), malodor-absorbing material, and combinations thereof. For example, antimicrobial agents can comprise cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, for example, zinc salts, and acids thereof, heavy metal salts of pyrithione, especially zinc pyrithione, zinc phenylsulfate, farnesol, and combinations thereof.

D. Cyclodextrin Complexing Material

Antiperspirant soft solid compositions can include a cyclodextrin complexing material for substantially hiding a fragrance material until a triggering mechanism can occurred, such as, for example, perspiration, to release the fragrance material. As used herein, the term “cyclodextrin” can includes any known cyclodextrins, such as unsubstituted cyclodextrins containing from about six to about twelve glucose units, especially alpha-cyclodextrin, beta-cyclodextrin, gamma-cyclodextrin, and/or their derivatives, and/or mixtures thereof. Suitable cyclodextrins can include cyclodextrins selected from the group consisting of beta-cyclodextrin, hydroxypropyl alpha-cyclodextrin, hydroxypropyl beta-cyclodextrin, methylated-alpha-cyclodextrin, methylated-beta-cyclodextrin, and mixtures thereof. Cyclodextrins can be included within antiperspirant soft solid compositions from about 0.1%, from about 1%, from about 2%, or from about 3% to about 25%, to about 20%, to about 15%, or to about 10%, by weight of the antiperspirant soft solid composition. Additional suitable examples of cyclodextrin complexing materials are described in U.S. Patent Application Publication No. 2008/0213204.

E. Optional Ingredients

Antiperspirant soft solid compositions can also include one or more optional ingredients which can modify physical or chemical characteristics of the compositions or serve as additional “active” components when deposited on the skin. Such optional ingredients can be included in an antiperspirant soft solid composition provided that the optional ingredients can be physically and chemically compatible and do not otherwise unduly impair product stability, aesthetics, or performance of the antiperspirant soft solid composition. Non-limiting examples of such optional ingredients can include, but are not limited to, pH buffering agents, additional malodor controlling agents, fragrance materials, emollients, humectants, soothing agents, dyes and pigments, medicaments, baking soda and related materials, preservatives, and soothing agents such as aloe vera, allantoin, D-panthenol, pantothenic acid derivatives (e.g., those disclosed in U.S. Pat. No. 6,495,149), avocado oil and other vegetative oils, and lichen extract.

### III. Rheology

An antiperspirant soft solid composition can have a select rheology profile that can help to improve product stability and performance. A rheology profile can include a combination of product hardness in the form of penetration force (gram-force), static yield stress (Pa) values, and/or high shear stress viscosity. Methods for determining such characteristics of the rheology profile are described in detail below.

The product hardness of the composition can be characterized as a penetration force value and generally describes the hardness of a composition prior to being dispensed from an applicator package (the package used by the consumer to apply the composition). When applicable, the penetration force of the composition can be from about 130 gram-force to about 500 gram-force; from about 165 gram-force to about 305 gram-force; from about 180 gram-force to about 250 gram-force, or any combination thereof.

Products with an insufficient static yield stress value can shear thin too much prior to application by an end user, or can otherwise be physically unstable, especially during extended storage in a closed applicator package or during rough shipping to distributors or consumers. Such product instability or excessive thinning of a product matrix can result in solvent syneresis from the composition during packaging, shipping, or extended storage. Exemplary static yield stress values are from about 100 Pa to about 3,000 Pa; from about 300 Pa to about 2,000 Pa; from about 500 Pa to about 1300 Pa; from about 500 Pa to about 1500 Pa, or any combination thereof.

A third rheology parameter is high shear viscosity. This parameter shows how a composition behaves under high shear. Products with a low high shear viscosity can fall apart and/or weep when they are dispensed through the porous applicator domes often used with soft solid products. Products with a high shear viscosity that is too high will be too difficult to dispense through the applicator. Exemplary high shear viscosity values include from about 2,000 Pa to about 50,000 Pa; from about 3,000 Pa to about 25,000 Pa; from about 5,000 Pa to about 20,000 Pa; or any combination thereof. Additionally, it is believed that within the exemplary values, there is a range at which the composition will exhibit additional stability. It is believed this additional stability at least partially contributes to an ability to use less wax as structurant in a composition and maintain acceptable syneresis. High shear viscosity values which can contribute to better stability include those from about 5,000 Pa to about 20,000 Pa.

A. Static Yield Stress/High Shear Viscosity

To determine static stress yield values for the antiperspirant soft solid compositions herein, a two-part test can be conducted. First, a controlled stress ramp can ramp up linearly, and can measure a shear rate at each point of stress. In the second part of the two-part test, a controlled shear rate ramp can be linearly increased and shear stress can be measured. A rheological model can be used to fit the data in both segments of the test, and a value can be determined from the rheological model for both segments.

Compositions are collected after they have been dispensed through their consumer use package and can be analyzed using a rheometer. In particular, the rheometer can be a Thermo Scientific Haake RheoStress 600 (available from TA Instruments, New Castle, Del., U.S.A) and data collection and analysis can be performed using rheology software, which can be RheoWin Software Version 2.84 or greater.

To prepare product samples, each product sample can be conditioned at about 23° C. until rheological properties can stabilize. An incubation period can be specified for each type of antiperspirant soft solid composition.

To operate the rheometer, parallel plates can be installed, and using the rheology software, a zero point for a gap between the parallel plates can be determined. A sufficient amount of the product sample can be loaded to ensure that entire serrated portions of the parallel plates can be in contact with the product sample once the product sample can be in a measurement position. A spatula can be used to carefully scrape dispensed product onto the serrated portion of a base plate. Once the product can be loaded, the rheology software can be used to move the parallel plates. A controlled stress ramp can be conducted followed by a controlled shear rate ramp.

Next, the rheology software can be used to determine shear yield stress values based on the controlled stress ramp and the controlled shear rate ramp. Data from the rheology test can be plotted as viscosity (Pa-s) on a log scale versus linear applied stress (Pa). “Static yield stress” refers to a point in a stress sweep analysis of a product at which point the rheometer is first capable of measuring product viscosity. The static yield stress is extrapolated from the data from a flow region along a shear rate measurement within 50-5001/s.

For the high shear viscosity, the shear rate measurement is done at 10 l/s instead of the 50-500 l/s used for the static yield stress.

B. Product Hardness

Antiperspirant soft solid compositions can be evaluated for product hardness (gram-force) and defined in terms of penetration force values. “Penetration force value” as used herein can represent a force required to move a standard 45° angle penetration cone through the composition for a distance of 10 mm at a rate of 2 mm/second. Values can be measured at 27° C. and 15% relative humidity using a TA-XT2 Texture Analyzer, available from Texture Technology Corporation, Scarsdale, N.Y., U.S.A. Higher values represent a harder product and lower values represent a softer product. The cone is available from Texture Technology Corp., as part number TA-15, and can have a total cone length of about 24.7 mm, an angled cone length of about 18.3 mm, and a maximum diameter of an angled surface of the cone of about 15.5 mm. The cone can have a smooth, stainless steel construction and weigh about 17.8 grams.

To operate the TA-XT2 Texture Analyzer, the cone, or probe, can first be attached to a probe carrier arm and cleaned with a low-lint wipe. Subsequently, a top stop and a bottom stop can be checked to ensure each is in a desired position. Test samples will generally be the composition inside the consumer applicator. Once samples have been properly prepared by removing any top portion of the container so that the cone or probe has access to the composition in the package, a product sample can be placed on a test base. A template can be used to ensure the product sample is at a desired location on the test base such that the cone can be in a position to contact the product sample at a midpoint between a canister side and a canister screw.

After the cone can be adjusted to about 1 cm above the product sample, a “RUN” button on the Texture Analyzer can be pressed. A measurement can be taken by the Texture Analyzer while a canister containing the product sample is held. The cone can take a measurement and automatically disengage from the product sample. Two measurements can be taken for each canister.

C. Weeping Score Test Method

Take an antiperspirant product in a consumer applicator package and remove the cap. Turn the handwheel on the applicator package in order to prime the package. Do so until antiperspirant composition is protruding from all of the apertures on the dome of the package. Then, wipe the dome clean of the composition. Repeat this for five more samples, for a total of six samples. After all of the samples are primed and clean, set a timer for 5 minutes. Once the 5 minutes has elapsed, look at each sample and give them a grade from 0-6. This is Grade 1. The grading scale is: 6—Liquid is present on sides of the dome and/or canister wall. 5-Liquid is pooling on the dome surface. 4—Liquid is pooling in inner and outer dome holes. 3—Liquid is pooling in outer dome holes and glossy dome surface. 2—Glossy product in dome holes and glossy dome surface. 1—Glossy product surface in dome holes. 0—Product surface is dry.

After all of the samples have been graded, repeat the priming process by turning the handwheel four clicks, or far enough to expel enough product through the apertures in the dome to get to fresh product, and wiping the domes clean. Once all domes are clean, then set the timer for five minutes. After the 5 minutes has elapsed, look at each sample and give them a grade from 0-6 using the scale above. This is Grade 2. Repeat this process one more time from priming to grading in order to get Grade 3. This constitutes three grades for the top portion of product in the canister.

This process should then be repeated for composition at both the middle and bottom portion of the canisters as well. Just dispense enough product to get you to the middle (or bottom portion) and repeat the steps above to take three grades at that level. This will result in three grades from the top portion, three grades from the middle portion, and three grades from the bottom portion, for a total of 9 grades. Report the average grade taken.

D. Vacuum Weeping Method

Take an antiperspirant product in a consumer applicator package and remove the cap. Turn the handwheel on the applicator package in order to prime the package. Do so until antiperspirant composition is protruding from all of the apertures on the dome of the package. Then, wipe the dome clean of the composition.

Wrap the outside of the canister in a liquid absorbing paper and tape in place. Remove and tare the paper, then replace back on the canister.

Place the canister into a Vacuum chamber at 0.5 atm for 1 hour. Liquid may run out of the canister down the side of the package and be absorbed by the paper.

Remove the canister from the chamber, remove the liquid absorbing paper and weigh it. Determine how much liquid was extracted out of the package and ran down the side of the canister by comparing the final weight of the liquid absorbing paper to the tare weight.

### IV. Methods

The compositions as described above also give rise to some useful methods. For example, one method includes a method of enhancing the stability of an anhydrous soft solid antiperspirant composition comprising formulating the composition to comprise a wax blend comprising a wax and at least two wax additives. Another method includes a method of enhancing the stability of an anhydrous soft solid antiperspirant composition comprising formulating the composition with a wax level of about 3% to about 6% by weight of the composition. An additional method includes a method of enhancing the stability of an anhydrous soft solid antiperspirant composition comprising formulating the composition to have a high shear rheology of about 5,000 Pa to about 20,000 Pa. Another method includes formulating an anhydrous soft solid composition to have a vacuum weeping score of about 0.10 g or less. All of the specifics of the compositions and their properties discussed above may be used herein in the methods.

### V. Examples

The example formulations included hereinafter can be prepared according to the following method. All ingredients other than active and perfume are put into a container and heated to 85° C. with mixing. Once the solids are completely dissolved, the temperature of the batch is brought down to 75° C. at which point the active and perfume are added. Once the active and perfume are incorporated, mill the composition for 5 minutes, cool it to below 60° C. and pour the product into the dispensing container. The product is placed in consumer dispensing containers where it is allowed to cool to room temperature.

Table 1 illustrates three formulations for antiperspirant soft solid compositions (Examples 1-3). For each formulation, tests were performed to determine penetration force values and values for static yield stress, which are also listed in Table 1.

TABLE 1

| Ingredient | Example 1, wt. % | Example 2, wt. % | Example 3, wt. % |
|---|---|---|---|
| Cyclopentasiloxane D-5 | 55.885-58.885 | 56.010-59.010 | 57.010-60.010 |
| Dimethicone | 5.000 | 5.000 | 5.000 |
| PPG-14 Butyl Ether | 0.500 | 0.500 | 0.500 |
| Petrolatum | 3.000 | 3.000 | 3.000 |
| Glyceryl Tribehenate | 4.500 | 2.500 | 2.000 |
| Sucrose Polybehenate | 1.125 | — | — |
| Polyethylene | 0.500 | 2.500 | — |
| Ozokerite | — | 1.000 | 1.000 |
| Synthetic Wax | — | — | 2.000 |
| Alum. Zirconium Trichlorohydrex Gly | 26.490 | 26.490 | 26.490 |
| Perfume | 0.000-3.000 | 0.000-3.000 | 0.000-3.000 |
| Penetration Force Value (gF) | 133 | 225 | 185 |
| Static Yield Stress (Pa) | 876 | 1,261 | 926 |

Table 2 illustrates three additional formulations for antiperspirant soft solid compositions (Examples 4-6). For each formulation, tests were performed to determine penetration force values and values for static yield stress, which are also listed in Table 2.

TABLE 2

| Ingredient | Example 4, wt. % | Example 5, wt. % | Example 6, wt. % |
|---|---|---|---|
| Cyclopentasiloxane D-5 | 59.500-62.500 | 60.750-63.750 | 62.500-65.500 |
| Dimethicone | 5.000 | 5.000 | 5.000 |
| Glyceryl Tribehenate | 5.000 | 2.500 | 1.700 |
| Sucrose Polybehenate | 1.250 | — | — |
| Polyethylene | 1.000 | 2.500 | — |
| Ozokerite | — | 1.000 | 0.850 |
| Synthetic Wax | — | — | 1.700 |
| Alum, Zirconium Trichlorohydrex Gly | 25.250 | 25.250 | 25.250 |
| Perfume | 0.000-3.000 | 0.000-3.000 | 0.000-3.000 |
| Penetration Force Value (gF) | 140 | 218 | 220 |
| Static Yield Stress (Pa) | 645 | 1,153 | 1,185 |

Table 3 illustrates another three additional formulations for antiperspirant solid compositions (Examples 7-9). For each formulation, tests were performed to determine penetration force values and values for static yield stress, which are also listed in Table 3.

TABLE 3

| Ingredient | Example 7, wt. % | Example 8, wt. % | Example 9, wt. % |
|---|---|---|---|
| Cyclopentasiloxane D-5 | 55.595-58.595 | 56.320-59.320 | 56.280-59.280 |
| Dimethicone | 5.000 | 5.000 | 5.000 |
| PPG-14 Butyl Ether | 2.000 | 2.000 | 2.000 |
| Petrolatum | 3.000 | 3.000 | 3.000 |
| Glyceryl Tribehenate | 4.500 | 2.250 | 3.680 |
| Sucrose Polybehenate | 1.125 | — | — |
| Polyethylene | 0.500 | 2.250 | — |
| Ozokerite | — | 0.900 | 0.800 |
| Synthetic Wax | — | — | 0.960 |
| Glucosamine | 0.010 | 0.010 | 0.010 |
| Panthenyl Triacetate | 0.010 | 0.010 | 0.010 |
| Tocopheryl Acetate | 0.010 | 0.010 | 0.010 |
| Alum. Zirconium Trichlorohydrex Gly | 25.250 | 25.250 | 25.250 |
| Perfume | 0.000-3.000 | 0.000-3.000 | 0.000-3.000 |
| Static Yield Stress (Pa) | 1,174 | 1,051 | 1,270 |

Table 4 illustrates yet another eight additional formulations for antiperspirant soft solid compositions.

TABLE 4

| Ingredient (wt. %) | Ex. 10 | Ex. 11 | Ex. 12 | Ex. 13 | Ex 14 | Ex. 15 | Ex. 16 | Ex. 17 |
|---|---|---|---|---|---|---|---|---|
| Cyclopentasiloxane D-5 | 57.00-60.00 | 57.50-60.50 | 56.07-59.07 | 55.45-58.45 | 54.80-57.80 | 59.05-62.05 | 55.55-58.55 | 56.25-59.25 |
| Dimethicone | 5.000 | 5.000 | 5.000 | 5.000 | 5.000 | 5.000 | 5.000 | 5.000 |
| PPG-14 Butyl Ether | 0.500 | 0.500 | 0.500 | 0.500 | 0.500 | 0.500 | 0.500 | 0.500 |
| Petrolatum | 3.000 | 3.000 | 3.000 | 3.000 | 3.000 | 3.000 | 3.000 | 3.000 |
| Glyceryl Tribehenate | 5.000 | 2.000 | 5.500 | 2.500 | 5.400 | 0.000 | 4.500 | 3.000 |
| Polyethylene | | 3.750 | | 2.400 | 1.600 | 3.400 | | |
| Synthetic Wax | | | 1.680 | 0.400 | 1.150 | | 2.900 | |
| Castor Wax | | | | 1.500 | | | | |
| Ozokerite | | | | 1.000 | 0.300 | 0.800 | 0.300 | 1.000 |
| Sucrose Polybehenate | 1.250 | | | | | | | |
| Beeswax | | | | | | | | 3.000 |
| Alum. Zirconium | 25.250 | 25.250 | 25.250 | 25.250 | 25.250 | 25.250 | 25.250 | 25.250 |
| Trichlorohydrex Gly | | | | | | | | |
| Perfumes | 0.0-3.0 | 0.0-3.0 | 0.0-3.0 | 0.0-3.0 | 0.0-3.0 | 0.0-3.0 | 0.0-3.0 | 0.0-3.0 |

TABLE 5

| | Commercial Example 1 | Comparative Example 1 | Comparative Example 2 |
|---|---|---|---|
| Raw Materials | Weight % | Weight % | Weight % |
| Cyclopentasiloxane D-5 | 62.000 | 62.000 | 62.000 |
| Dimethicone | 5.000 | 5.000 | 5.000 |
| Glycerol Tribehenate | 5.000 | 5.000 | 5.000 |
| C18-36 Acid Triglyceride | 1.250 | — | — |
| Polyethylene | — | 1.250 | — |
| Synthetic Wax | — | — | 1.250 |
| AP Active | 25.250 | 25.250 | 25.250 |
| Perfume | 1.500 | 1.500 | 1.500 |
| Total | 100.000 | 100.000 | 100.000 |
| Weeping Score | 0.46 | 1.09 | 0.87 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All documents cited in the Detailed Description are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An antiperspirant soft solid composition comprising a wax blend, the wax blend comprising:
a) from about 0.1% to about 6.0% of glyceryl tribehenate, by weight of the antiperspirant soft solid composition;
b) from about 0.1% to about 5.0% of a first wax additive, by weight of the antiperspirant soft solid composition, the first wax additive comprises one of a monoglyceride, a polymeric thickener, or an inorganic thickener; and
c) from about 0.1% to about 5.0% of a second wax additive, by weight of the antiperspirant soft solid composition, wherein the second wax additive comprises one of the remaining monoglyceride, polymeric thickener, or inorganic thickener, such that the first wax additive and the second wax additive are different;
wherein the antiperspirant soft solid composition is substantially free of a C₁₈-C₃₆ triglyceride and exhibits a static yield stress value from about 100 Pa to about 3,000 Pa.

2. The antiperspirant soft solid composition of claim 1, wherein the wax blend further comprises a third wax additive.

3. The antiperspirant soft solid composition of claim 1, wherein the wax blend comprises:
a) from about 0.5% to about 5.5% of glyceryl tribehenate, by weight of the antiperspirant soft solid composition;
b) from about 0.3% to about 3.8% of the first wax additive, by weight of the antiperspirant soft solid composition; and
c) from about 0.3% to about 3.8% of the second wax additive, by weight of the antiperspirant soft solid composition.

4. The antiperspirant soft solid composition of claim 3, wherein the first wax additive comprises an inorganic thickener comprising ozokerite.

5. The antiperspirant soft solid composition of claim 3, wherein the second wax additive comprises a polymeric thickener comprising polymethylene or polyethylene.

6. The antiperspirant soft solid composition of claim 1 exhibiting a penetration force value from about 180 gram-force to about 250 gram-force.

7. The antiperspirant soft solid composition of claim 6 exhibiting a static yield stress value from about 600 Pa to about 1,500 Pa.

8. The antiperspirant soft solid composition of claim 1, wherein the antiperspirant soft solid composition comprises from about 3% to about 8.5%, by weight of the antiperspirant soft solid composition, of the wax blend.

9. The antiperspirant soft solid composition of claim 1 further comprising a liquid carrier and an antiperspirant active.

10. The antiperspirant soft solid composition of claim 1, wherein the composition is anhydrous.

11. The antiperspirant soft solid composition of claim 1, wherein the first wax additive comprises from about 1.0% to about 1.5% of the monoglyceride, by weight of the antiperspirant soft solid composition.

12. The antiperspirant soft solid composition of claim 11, wherein the monoglyceride is sucrose polybehenate.

13. The antiperspirant soft solid composition of claim 1, wherein the composition has a penetration force value from about 130 gram-force to about 500 gram-force.

14. The antiperspirant soft solid composition of claim 1, wherein the composition has a high shear viscosity of about 5,000 Pa to about 20,000 Pa.

15. An antiperspirant soft solid composition, comprising:
1) from about 3% to about 8%, by weight of the composition, of a wax blend comprising
a) from about 1% to about 6%, by weight of the composition, of glyceryl tribehenate;
b) from about 0.5% to about 2.5%, by weight of the composition, of ozokerite;
c) from about 0.3% to about 3.5%, by weight of the composition, of polymethylene; and
2) from about 55% to about 75%, by weight of the composition, of a liquid carrier;
wherein the composition is substantially free of a C₁₈-C₃₆ triglyceride and microcrystalline wax.

16. The composition of claim 15, wherein the polymethylene has a melting point of about 65° C. to about 75° C.

17. The composition of claim 16, wherein the composition is anhydrous and/or free of castor wax.

18. The composition of claim 17, wherein the composition has a high shear rheology of about 5,000 Pa to about Pa.

19. An anhydrous soft solid antiperspirant composition, comprising:
1) from about 3% to about 6.5%, by weight of the composition, of a wax blend consisting essentially of:
a) from about 1.5% to about 2.5%, by weight of the composition, of glyceryl tribehenate;
b) from about 1.5% to about 2.5%, by weight of the composition, of ozokerite;
c) from about 0.75% to about 1.25%, by weight of the composition, of polymethylene with a melting point of about 65° C. to about 70° C.; and
2) from about 55% to about 75%, by weight of the composition, of a liquid carrier.
